(19) United States
(12) Patent Application Publication
(10) Pub. No.: US 2025/0278441 A1
(43) Pub. Date: Sep. 4, 2025

(54) Automated Prescription Drug Routing Software

(71) Applicant: TrackRecord Health, Inc., Wake Forest, NC (US)

(72) Inventors: Andrew Chan, Wake Forest, NC (US); Sean Cannon, Wake Forestt, NC (US); Leo Spector, Wake Forest, NC (US)

(21) Appl. No.: 18/591,800

(22) Filed: Feb. 29, 2024

(51) Int. Cl. G06F 16/953; G16H 20/10

(52) U.S. Cl. CPC G06F 16/953; G16H 20/10

(57) ABSTRACT

This document presents a system for optimized the search and acquisition of multiple medication pharmacies, manufacturer's sites, and other web pages for discount and coupon information for medications of interest to a user. The search algorithm disclosed can determine whether it is more advantageous to purchase prescription medications online or via one or more physical retail locations. The system may also determine and present to the user multiple locations with optimized prices or coupons from which the user may select for fulfillment of their medications.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

Single search engines that are used to search for drug or medication pricing on the web or in networked sites may provide a process that crawls multiple sources for the best price on a purchase. These web crawler search engines may aggregate, filter, and sort search results for particular drugs or medications. Existing systems may provide the ability to capitalize on the best price for a singular purchase of a drug or medication. Existing systems may also provide a listing of prices through geolocation to show physical locations for particular drug or medication pricing. However, most existing systems do not permit the acquisition through a transfer of a medication or drug directly from one medical fulfillment center to another utilizing the existing platform.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of prescription price comparison and optimization consistent with certain embodiments of the present invention.

FIG. 2 is a view of prescription transfer capability consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The innovation, the ScriptScouts system, presents a novel and proprietary set of search algorithms to crawl the web for the best prices and pickup locations, such as physical retail or online pharmacy locations, for prescription medications. The ScriptScouts system provides a subscription service to provide users with a monthly update on the best pickup locations or online pharmacies for the user's medications. The monthly updates may be provided through emailed PDF, email, text notification, or a web user interface communications.

The ScriptScout system application is available to users via a web interface, mobile application, and through a REST API layer. The service provided by the ScriptScouts system seamlessly transfers prescriptions between pharmacies on behalf of the user through a user authorized medical release. The ScriptScouts optimize cash-pay prices, cash prices relative to insurance to help the user optimize cash/Health Saving Account (HSA)/Flexible Spending Account (FSA) spend relative to their deductible and out-of-pocket maximum.

The ScriptScouts system presents a novel search engine algorithm that focuses on searches for both brand name and generic pharmaceutical drugs. The searches may be based on drug name, quantity, form, and dosage and may search for one or more medications simultaneously. Medication information is stored in a secured manner. Based upon search results an electronic routing system permits prescriptions to be routed to the best physical retail location or online pharmacy for pickup and/or delivery. The system continuously scans all targeted web sources for changes in drug prices. User notifications and prescription transfers are initiated as needed based upon any notified changes in drug prices being monitored by the system. The system may provide coupon/discount information on prescription medications, including information that is required for pharmacies to process discounts when checkout and payment for any medication.

Prescription drug prices may vary up to 90% between sources. The system may identify the purchase location with the lowest price in a short time period, such as, in a non-limiting example, in less than 5 seconds. The Wholesale Acquisition Cost (WAC) paid by pharmacies for prescription medications can vary on a weekly basis which places a burden on consumers in placing a requirement on a consumer to continually monitor and shop for the best price at any given time. The system herein described automates this monitor and shopping process to locate and report the best price for a medication to a user in real time.

Additionally, transferring prescriptions between pharmacies is often a difficult and time consuming process often requiring multiple doctor visits and phone calls. The system herein described automates the prescription transfer process to that a user is continually receiving the best price on any medication being monitored by the system.

Thus, the system presents an ability for a user to always receive the lowest or most effective price for searched prescription medications. The system also eliminates the hassle of shopping for medications and transferring prescriptions between pharmacies. The system also permits a user to determine whether it is optimal to purchase medications from a physical retail pharmacy or an online pharmacy.

The system creates and operates a search engine that can simultaneously search multiple drug discount sites, such as, in non-limiting examples, GoodRx, SingleCare, Amazon Pharmacy, CostPlus Drugs, among other sites, manufacturer's websites, online pharmacies, and other web pages for discount/coupon information. As a result of the search, the search engine may present options to a user via a user interface for both brand name and generic drugs. The search engine may determine whether it is more advantageous to purchase prescription drugs online or via physical retail pharmacy. The system may use geolocation to maximize an individual's savings when purchasing prescription drugs.

The invention also provides an automated system that facilitates requests, and digital transfers of, prescriptions between a doctor's office and a pharmacy based on the search engine search results. The system has the ability to route all prescriptions to a single pharmacy or route multiple prescriptions to multiple pharmacies. Additionally, the invention presents an automated system that determines, based upon the search engine results, whether it is advantageous to pay cash or pay through insurance coverage to purchase a particular medication based on the best pickup/order location.

The system presents a workflow that permits a user to search for a medication or drug by name, using voice or typed text, or using a camera to scan a bottle utilizing Optical Character Recognition (OCR). The system may then process the drug name and determine whether the entered drug or medication name is a brand name drug or a generic drug or medication. The user may also enter information on the medication or drug dosage, form, and quantity of the medication.

The user may indicate the scope of the search as covering physical pharmacies, online pharmacies, or both. The system, active within a data processor within a network server, will then determine, based upon the type of drug or medication, whether it is tenable to search online pharmacies. For certain medications or drugs online searching may not be permitted, such as, in a non-limiting example, medications such as Amphetamine Salts cannot be ordered online.

Upon entering the drug or medication name information, the system may initiate a web search on designated drugs or medications. The web search may be initiated by a user to query the ScriptScouts medications database for previously-scraped data, or it may be initiated via robotic process automation, which may or may not be a serverless function, such as an Amazon Web Services (AWS) Lambda function, or an adjacent or equivalent technology. This technology parses the HTML, XML, JSON, or any other available format of data provided by target pages and identifies and updates collected and persisted data such that the system May offer the operational search algorithm the most up-to-date pricing and discounts that may be available online. If an API is available, the system may also initiate an API call to retrieve data. This retrieved data, inclusive of drug prices based on quantity, form, and dosage, are stored in a time series database alongside corresponding pick-up or order location. Coupon and/or discount information is also retrieved and stored in the database maintained in the electronic storage associated with the system. The coupon/discount information may consist of BIN numbers, PCN numbers, and other identifying information required to process a discount or coupon at a physical retail pharmacy upon check out.

The system provides a font end web application that displays the search results to the user and allows the user to sort and filter the results. Coupon and/or discount information is displayed alongside pick up location information and the estimated the drug price. Users can download the discount and/or coupon information via pdf, excel file, or any digital wallet.

Upon user selection which discount and/or coupon presented to the user that they may want to use for their medications or drugs, the user may use an automated backend system to request that a prescription be sent on their behalf to a designated physical retail pharmacy and/or online pharmacy. This request may be initiated via a phone call, an email, or an API request through an electronic healthcare record system. If the patient has a physical or digital copy of their prescription, the patient as a user may upload the copy of the prescription through the user interface and the system may designate which pharmacies to which it should be sent for fulfillment.

Upon completion of the initial search for discounts and coupons, the user's preferences may be saved in each user profile along with the medication list for each user and may be stored in a database maintained within the electronic storage associated with the system. On a biweekly or monthly basis, the web search engine application may continuously scan the web and other networks and provide a communication to a user to update each user if a better price, discount, or pick up location is found. The communication update to each user may be transmitted via email, text, or within the search engine application during a search operation. The continuous scanning process uses the same technologies used to crawl the web and other networks as previously described.

The search engine algorithm optimizes for obtaining the best price for a prescription drug whether it is through insurance payments or paid for in cash. The algorithm also optimizes whether it is most beneficial to pick up the medications using s coupon at a retail pharmacy or purchasing directly from an online pharmacy. The search engine algorithm also optimizes for the best price based upon the quantity purchased and the difference in price between brand name drugs and generics.

The search engine algorithm may gather data from sources using any of three methods. Those methods consist of 1) an excel sheet, or equivalent data form, that is scripted into the database, 2) data extracted from one or more 3^{rd} party data sources through an API integration, and 3) data may be extracted via web scraping and Chat GPT or other generative AI large language models that are applied to raw HTML for discovered web and networked pages to convert input data to data structures that are database compatible.

In an embodiment, the use of large language models in conjunction with web scraping permits python based open source packages to be used to glean raw HTML from publicly available websites. An external database of drug and medication names and their corresponding possibilities for dosages, quantities, and form are integrated with data in one or more large language models. Large language models may be trained to recognize words that are synonyms for the following prescription drug information. In non-limiting examples, dosage may be equated to the term “strength”, quantity may be equated to the term “count”, form may be equated to the term “type”, and other synonyms may be discovered and used within the search engine algorithm.

In an embodiment, a statistical probability may be assigned to words extracted from HTML that are not an exact match for the aforementioned words. In the event that a probability of being a synonym is greater than 90% probable, a user may manually verify the validity of the synonym prior to it being transformed into a data structure that is compatible with the database. A library of synonyms is created and maintained as new sources of HTML are fed into the large language models.

The search engine algorithm may optimize payment logic by first determining if the user is paying for a medication or drug utilizing an insurance payment, or payment through cash. If the payment is made through an insurance policy, the search engine algorithm ceases the price optimization. If the payment is to be made by cash or electronic payment, the search engine algorithm payment optimization algorithm may determine if there is a coupon available. If there is no coupon or discount available or if the user is paying an online pharmacy for the drug or medication, the optimization algorithm is satisfied and the process is complete. If there is a cash pay coupon available to the user the optimization algorithm may determine user's preference for a single pharmacy or multiple pharmacies for price optimization when using the coupon. The user may then select the presented pharmacy or pharmacies for the selection of where to acquire the medication or drug based upon the coupon price.

If approved, an automated email message is sent to a provider's email box. Alternatively, if the system provides an integration with a client relationship management platform, the patient data is synchronized with the platform and employees may manually call the medical practice, or the system may trigger an automated calling system to reach out on behalf of the patient to complete the transfer activity.

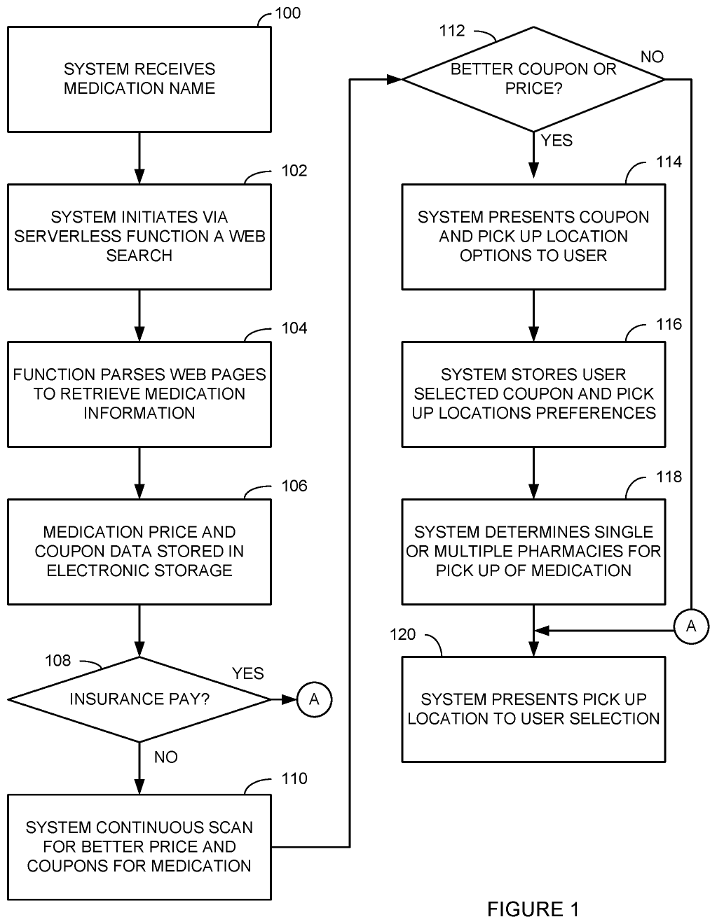

Turning now to FIG. 1, this figure presents a view of prescription price comparison and optimization consistent with certain embodiments of the present invention. In an exemplary embodiment, at 100 the system may receive the medication name, as well as details about the medication such as dosages, quantities, and form of the medication to be dispensed. At 102 the system may initiate a search for the prescription for which the name has been provided across a plurality of sources including web resources and other published sources. At 104 the search function algorithm may parse the discovered web pages and other discovered sources to retrieve information about the medication based upon the name provided. At 106 the search function algorithm may store the medication price and any coupon data associated with the medication into a database in electronic storage associated with the user, or patient, and the medication or medications for which an optimized price is being searched and evaluated. At 108 the system determines whether payment for the medication will be made by cash or other direct payment, or whether the payment for the medication will be made by an insurance provider. If the medication payment is to be made by an insurance provider, the system accepts the price that the insurance provide will pay for the medication as the final price and presents the pick-up location to the user as the location at which the user may pick up the medication after fulfillment at 120.

In an embodiment, if the payment is to be made in cash or other form of direct payment at 110 the system search algorithm initiates a continuing scan of all network, public, and private sources for the best price and/or coupons for reduction in price for the medication to be acquired. At 112, the system attempts to determine if a better price or coupon for a better price has been discovered. If a coupon or better price has been discovered, at 114 the system may present the price, coupon, and pick up location to a user. At 116, the system receives and stores the user selection for price and/or a coupon as well as the preferred pick-up location as selected by the user. At 118 the system may also determine single or multiple fulfillment locations, such as pharmacies or other fulfillment locations, and provide these choices to the user for pick-up locations. Once again, at 120, the system presents the pick-up location or locations to the user for the user's action in fulfillment.

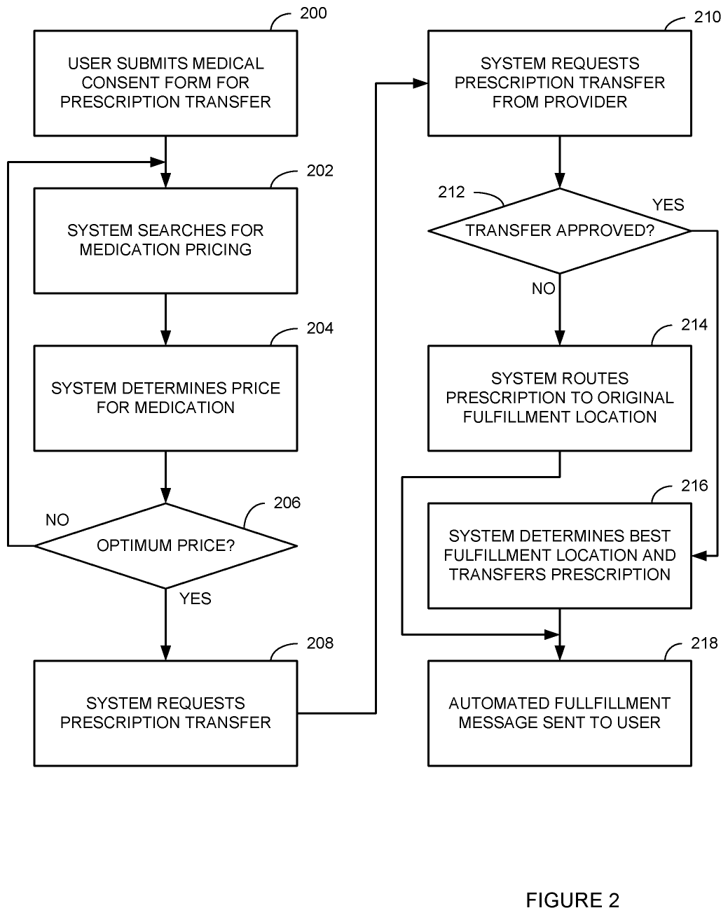

Turning now to FIG. 2, this figure presents a view of the prescription transfer capability consistent with certain embodiments of the present invention. In an exemplary embodiment, the system also may permit the automated transfer of a prescription from one pharmacy or provider to another. To participate in this activity a user must submit a medical consent form at 200. After submission of the form the system may perform a search for the optimal pricing for the medication(s) named in the request at 202. Upon conclusion of the search, one or more medication prices are discovered at 204 and the optimal price determination is made at 206, and the system facilitates an automated or manual transfer process for the prescription. At 208, to facilitate this transfer process an API level integration may permit the application to request a prescription transfer using web protocols, or, alternatively, the system may act as middleware between an electronic healthcare record system, a digital prescription management system, and a pharmacy. The request may include the prescription name, quantity, dosage, and form. The search engine algorithm may send a request to have a prescription transferred at 210, the provider would grant approval at 212. If the provider does not grant approval for the prescription transfer, the system routes the prescription for fulfillment to the existing fulfillment partner of record at 214. If the provider does grant permission, the system determines the best fulfillment location for transfer of the prescription at 216, and the prescription would be automatically routed to the best pharmacy as determined by the search engine algorithm at 218.

While certain illustrative embodiments have been described, it is evident that many alternatives, modifications, permutations and variations will become apparent to those skilled in the art in light of the foregoing description.

1. A system for optimizing medication acquisition, comprising:
a data processor receiving detailed information about a medication utilizing a medication name;
said data processor initiating a search function algorithm to perform a search across a plurality of sources for said medication name through accessing scripted database information, extracting data from third party sources connected to the algorithm through an Application Programming Interface (API), and/or utilizing a generative AI function to glean raw HTML from publicly available websites for said medication names and prescription drug information;
said search function algorithm utilizing a trained large language model to recognize synonyms for prescription drug information for said medication name to discover and use said synonyms in the medication name and drug information search;
said search function algorithm parsing said sources and the trained large language model to retrieve information about said medication name and drug information;
said data processor determining payment for the medication represented by said medication name;
said data processor determining that the payment for the medication discovered is a direct cash payment;
when a direct case payment is determined, said search function algorithm scanning all network, public, and private sources for best price and coupons for reduction of said best price;
said search function algorithm performing an updated search for said medication name, and determining user's preference for a single pharmacy or multiple pharmacies for price optimization when using a coupon;
said search function algorithm performing a price optimization action by transferring said best price, whether said best price is updated or not updated, to one or more pick-up locations or pharmacies acceptable to the user; said data processer presenting the best price, any coupon, and the pick-up location to which said best price was transferred to said user.

2. The system of claim 1, where said medication information comprises medication dose, quantity, and form information.

3. The system of claim 1, where said pick-up location comprises more than one pick up location as selected by the user.

4. The system of claim 1, where said best price transfer is performed utilizing web protocols to request and perform a medication transfer to a particular pick-up location.

5. The system of claim 1, where said data processor acts as middleware function between an electronic healthcare record system, a digital prescription management system, and a pharmacy to perform a medication transfer.

6. The system of claim 1, further comprising a request sent by the search engine algorithm to have a prescription for a selected medication transferred to a medical provider.

7. The system of claim 1, where the data processor routes the prescription for fulfillment to the existing fulfillment partner of record.

8. The system of claim 1, where the prescription for said medication is automatically routed to the best pharmacy location for pick up as determined by the search engine algorithm.
